(19) United States
(12) Patent Application Publication
(10) Pub. No.: US 2007/0104695 A1
(43) Pub. Date: May 10, 2007

(54) Breast augmentation and reconstruction system

(76) Inventors: Rodolfo C. Quijano, Laguna Hills, CA (US); Hosheng Tu, Newport Beach, CA (US); Kenneth J. Williams, Brawley, CA (US); Robert L. Carter, Joplin, MO (US); Alexander Kiselyov, Del Mar, CA (US)

Correspondence Address: HOSHENG TU, 15 RIEZ, NEWPORT BEACH, CA, 92657-0116, US

(21) Appl. No.: 11/490,431

(22) Filed: Jul. 20, 2006

Related U.S. Application Data
(63) Continuation-in-part of application No. 11/268,432, filed on Nov. 7, 2005.

(51) Int. Cl. A61K 35/12; C12N 5/08

(52) U.S. Cl. 424/93.7; 435/366

(57) ABSTRACT

A breast template for delivering stem cell formulation to a breast defect of a patient for treating or augmenting a breast tissue defect comprising a flexible band with at least one throughput hole for guiding an injecting needle to penetrate into the breast of the patient, wherein the flexible band is configured to be placed intimately against a surface of the breast.

### CROSS-REFERENCE TO RELATED APPLICATIONS

This application is a continuation-in-part application of U.S. patent application Ser. No. 11/268,432, filed Nov. 7, 2005, entitled “Breast Augmentation System,” the entire contents of the co-pending application are incorporated herein by reference.

### FIELD OF THE INVENTION

The present invention is related to stem cells for treatment of breast tissue defect, more particularly, the present invention relates to stem cells delivery system thereof to repair, augment or reconstruct a breast tissue defect in a patient.

### BACKGROUND OF THE INVENTION

It was reported that adipose-derived stem cells might be engulfed in injured heart muscle following a heart attack-like injury. Adipose, also known as fat tissue, contains a specialized class of stem cells, which are comprised of multiple cell types that might promote healing and repair. It appears that adipose-derived stem cells home in on specific sites of injury through biological signaling that occurs naturally during heart attacks.

In addition to pluripotent stem cells of embryonic origin, several groups described mammalian multipotent stem cell populations that are obtained from adult somatic cell sources. Non-embryonic multipotent stem cells include, for example, neural stem cells, mesenchymal stem cells, bone marrow stem cells and stem cells obtained from liposuction. It is important to note that the adult multipotent stem cells described in the prior art have limited potential, in that they have not been demonstrated to give rise to any and all cell types of the body. In general, a stem cell shows ability of a clonal stem cell population to self-renew, ability of a clonal stem cell population to generate a new, terminally differentiated cell type in vitro and ability of a clonal stem cell population to replace an absent terminally differentiated cell population when transplanted into an animal depleted of its own natural cells.

Mesenchymal stem cells are adult multipotent cells derived from multiple sources, including bone marrow stroma, blood, dermis, and periosteum. These cells can be cultured continuously in vitro without spontaneous differentiation. However, under the proper conditions, mesenchymal stem cells can be induced to differentiate into cells of the mesenchymal lineage, including adipocytes, chondrocytes, osteocytes, tenocytes, ligamentogenic cells, myogenic cells, bone marrow stroma cells, and dermogenic cells (U.S. Pat. No. 5,736,396). It was reported that mesenchymal cells, upon injection into either mouse or rat brains, are capable of migrating through the brain, engrafting, surviving, and differentiating into astrocytes, ependymal cells, or neurons, suggesting the capacity of mesenchymal stem cells to give rise to cells of a non-mesenchymal lineage (U.S. Pat. No. 5,197,985, No. 5,226,914, No. 5,486,359, and No. 5,736,396).

U.S. Pat. Nos. 6,429,013 and 6,841,150, entire contents of which are incorporated herein by reference, discloses pluripotent stem cells generated from adipose tissue-derived stromal cells and uses thereof. Specifically, the patents disclose that an isolated adipose tissue derived stromal cell is induced to express at least one characteristic of a neuronal cell, an astroglial cell, a hematopoietic progenitor cell, and a hepatic cell. Further, the patents discloses a method for dedifferentiating isolated adipose tissue-derived stromal cells, comprising: plating the isolated adipose tissue-derived stromal cells at a density of approximately 1,000 to 500,000 cells/cm² and incubating the cells in medium comprising i) serum; ii) at least one compound selected from the group consisting of: growth factors, hormones, cytokines and serum factors; and iii) optionally, an embryonic extract.

U.S. Pat. No. 7,015,037 B1, entire contents of which are incorporated herein by reference, discloses isolated stem cells of non-embryonic origin that can be maintained in culture in the undifferentiated state or differentiated to form cells of multiple tissue types. Particularly, the invention discloses an isolated cell population derived from human bone marrow, wherein the cells of the cell population co-express CD49c, CD90 and telomerase or wherein the cells of the population differentiate into cell types of at least two of ectodermal, endodermal, or mesodermal lineages.

Labat et al. in U.S. patent application publication no. 20060074044, entire contents of which are incorporated herein by reference, discloses stem cell growth factor-like polypeptides and isolated polynucleotides encoding such polypeptides, including recombinant DNA molecules, cloned genes or degenerate variants thereof especially naturally occurring variants such as allelic variants, antisense polynucleotide molecules, and antibodies that specifically recognize one or more epitopes present on such polypeptides, as well as hybridomas producing such antibodies.

Important parts of the breasts include mammary glands, the axillary tail, the lobules, Cooper's ligaments, the areola and the nipple. As breasts are mostly composed of adipose tissue, their size can change over time if the woman gains or loses weight. Adipose tissue is an anatomical term for loose connective tissue composed of adipocytes. Its main role is to store energy in the form of fat, although it also cushions and insulates the body. It has an important endocrine function in producing hormones such as leptin, resistin and TNF-α. It also functions as a reservoir of nutrients. Adipose tissue has an “intracellular matrix,” rather than an extracellular one. Adipose tissue is divided into lobes by small blood vessels. The cells of this layer are adipocytes.

Recent advances in biotechnology have allowed for the harvesting of adult stem cells from adipose tissue, allowing stimulation of tissue regrowth using a patient's own cells. The use of a patient's own cells reduces the chance of tissue rejection.

Five stages of breast development include: a) the first childhood stage: the breasts are flat and show no signs of development; b) the second breast bud stage: milk ducts and fat tissue form a small mound; c) the third breast growth stage: breast become rounder and fuller; d) the fourth stage with nipple and areola forming separate small mound: not all girls go through this stage; and e) the firth stage: breast growth enters finial stage showing an adult breast full and round shaped. For those women with breast defect, it is desirable to transplant stem cells or stem-cell-seeded porous scaffold as an implant to repair or augment the breast tissue defect.

Whereas embryonic stem cells are the building blocks for all of the cell types in the body, adult stem cells are a more specialized type of progenitor cell. Adult stem cells are found in specific tissues and have the ability to regenerate themselves, as well as differentiate into all of the cell types found in that tissue. The specific differentiation pathway that these cells enter depends upon various influences from mechanical influences and/or endogenous bioactive factors, such as growth factors, cytokines, and/or local microenvironmental conditions established by host tissues. Using cells from the developed individual, rather than an embryo, as a source of autologous or allogeneic stem cells would overcome the problem of tissue incompatibility associated with the use of transplanted embryonic stem cells, as well as solve the ethical dilemma associated with embryonic stem cell research.

There are several forms of fat in a human body: brown or fetal fat, common in youth, less so in adults but still present, visceral fat around internal organs, an endocrine organ in and of itself to be sure associated with leptin, IL6, TNF alpha along with about ten other factors not necessarily regarded as hormones, for example, TNF alpha (tumor necrosis factor-α), and abdominal wall subcutaneous fat which generally is not thought to share the endocrine features so associated with visceral fat.

Fat injections in the breasts have always been taboo, because large globules of fat can calcify and resemble cancerous tumors on mammograms. Recently, it was reported that microdroplets of autologous fat was injected to the breast using tiny needles after priming breasts with a suction device to increase blood supply. Six months later, MRIs found 90% of the fat still present with only minimal calcification, which was detectable as noncancerous. Early and adequate revascularization could be a factor in breast tissue regeneration.

Adipose tissue offers a potential source of multipotential stromal stem cells. Adipose tissue is readily accessible and abundant in many individuals. Obesity is a condition of epidemic proportions in the United States, where over 50% of adults exceed the recommended BMI based on their height. Adipocytes can be harvested by liposuction on an outpatient basis. This is a relatively non-invasive procedure with cosmetic effects that are acceptable to the vast majority of patients. It is well documented that adipocytes are a replenishable cell population. Even after surgical removal by liposuction or other procedures, it is common to see a recurrence of adipocytes in an individual over time. This suggests that adipose tissue contains stromal stem cells that are capable of self-renewal.

### SUMMARY OF THE INVENTION

One object of the invention is to provide a method and compositions for directing adipose-derived stromal cells cultivated in vitro to differentiate into breast tissue lipocyte stem cells derived from subcutaneous fat cells or the fat stem cells with enough angiogenesis factors for implantation into a recipient for the therapeutic treatment of pathologic conditions in breast tissue.

Some aspects of the invention relate to a method of providing stem cells for treatment of breast tissue defect. In one preferred embodiment, the method comprises providing stem-cell-seeded porous scaffold or construct as an implant to repair or augment a breast tissue defect in a patient. The adipose-derived stem cells home in on specific sites of breast defect or injury through biological signaling that occurs naturally for a breast defect or pathologic conditions.

Some aspects of the invention relate to a method of providing stem cells for cosmetically modifying breast tissue, wherein the method comprises providing 3D stem-cell-seeded scaffold or construct as an implant to cause breast tissue defect due to implantation and providing breast tissue regeneration through stem cells of stem-cell-seeded scaffold or construct for repairing or augmenting the breast tissue defect in a patient.

Some aspects of the invention relate to a method of treating a breast defect in a patient, the method comprising differentiating an isolated human adipose tissue derived stromal cell into subcutaneous fat stem cells with enough angiogenesis factors and administering the fat stem cells with enough angiogenesis factors to a breast defect area in the patient. In one embodiment, the fat stem cells with enough angiogenesis factors further comprises a biocompatible shaped matrix or scaffold, wherein the biocompatible matrix may be non-biodegradable or biodegradable. In a further embodiment, the biodegradable matrix may be made of a material selected from a group consisting of polymers or copolymers of lactide, glycolide, caprolactone, polydioxanone, trimethylene carbonate, polymers or copolymers of polyorthoesters and polyethylene oxide, and polymers or copolymers of aliphatic polyesters, alginate, cellulose, chitin, chitosan, collagen, copolymers of glycolide, copolymers of lactide, elastin, fibrin, glycolide/l-lactide copolymers (PGA/PLLA), glycolide/trimethylene carbonate copolymers (PGA/TMC), glycosaminoglycans, and hydrogel. In a further embodiment, the biocompatible matrix comprises a material selected from a group consisting of alginate, agarose, fibrin, collagen, methylcellulose, and combinations thereof.

In one embodiment, the breast defect is traumatically created by any of the following conditions or processes: inserting the biocompatible matrix into the patient, lumpectomy, mastectomy, breast reconstruction, breast injury, or other breast surgical procedures.

In an alternative embodiment, the fat stem cells with enough angiogenesis factors further comprises a biocompatible cell carrier, wherein the cell carrier may be in a form selected from a group consisting of slurry, gel, colloid, solution, or suspension that is flowable. In one embodiment, the cell carrier or gel is malleable. Further, the cell carrier is selected from a group consisting of alginate, agarose, fibrin, collagen, chitosan, gelatin, elastin, and combinations thereof. In one embodiment, the biocompatible cell carrier is biodegradable. The subcutaneous fat stem cells with enough angiogenesis factors is to stimulated a viable sustainable graft that won't develop avascular necrosis nor turn up with cancer sometime down the road.

Some aspects of the invention relate to a method of treating a breast defect in a patient, the method comprising differentiating an isolated human adipose tissue derived stromal cell into a breast tissue stem cell and administering the breast tissue stem cell to a breast defect area in the patient, wherein following administration of the stem cell to a breast defect area in the patient, the stem cell further differentiates in situ in the patient.

Some aspects of the invention provide a composition for treating a breast defect of a patient, comprising stem cells derived from adipose tissue and a temperature-sensitive cell carrier, wherein the stem cells may comprise breast tissue progenitor cells or subcutaneous fat stem cells with enough angiogenesis factors. In one embodiment, the temperature-sensitive cell carrier is methylcellulose, poly(N-isopropyl acrylamide), or the like. In one embodiment, the temperature-sensitive cell carrier is characterized by a first solution phase at a lower temperature and a second gel phase at a higher temperature. In another embodiment, the temperature-sensitive cell carrier is characterized by an expanded conformation at a lower temperature and a collapsed conformation at a higher temperature. In a further embodiment, the composition is a compressible foam, a shaped scaffold, a porous matrix or flowable/malleable material.

Some aspects of the invention provide a breast matrix system for treating a breast defect of a patient, comprising an implantable breast matrix and stem cells component, wherein stem cells are derived from adipose tissue. In one embodiment, the breast matrix comprises a fishbone configuration, the fishbone-configured breast matrix being characterized by an expandable construct with a plurality of close cells formed between longitudinal elements and connecting transverse elements. In another embodiment, the breast matrix system further comprises a delivery instrument for delivering the fishbone-configured breast matrix to a breast of the patient for treating the breast defect.

In one embodiment, the breast matrix of the breast matrix system of the present invention comprises an umbrella configuration, the umbrella-configured breast matrix being characterized by a plurality of radially expandable extending elements, each extending element having a distal end and a proximal end, wherein the proximal ends from all extending elements are secured together at one point. In another embodiment, the breast matrix system further comprises a delivery instrument for delivering the umbrella-configured breast matrix to a breast of the patient for treating the breast defect.

In one embodiment, the breast matrix of the breast matrix system of the present invention comprises a wrap-around configuration, the wraparound-configured breast matrix being made of shape memory material and characterized by a first pre-implant low-profile configuration at a lower temperature and a second implanted configuration at a higher temperature. In a further embodiment, the shape memory material is biodegradable polymer or Nitinol.

In one embodiment, the breast matrix of the breast matrix system of the present invention comprises a yo-yo configuration, the yoyo-configured breast matrix being characterized by a plurality of circular rings with varying diameters, wherein at least two circular rings are releasably secured to each other by a circular semi-ring to form an overall bowl-like configuration.

In one embodiment, the breast matrix is biodegradable or bioresorbable. In another embodiment, the breast defect is traumatically created by a process of inserting the breast matrix into a breast of the patient. In still another embodiment, the stem cells portion comprises breast tissue progenitor cells. Scarring is a major problem in the breast reconstruction application and it comes from fat necrosis. What one would want to inject would be lipocytes or adipocytes that are stromal only with no ductal or lobular developmental potential.

In one embodiment, the breast matrix system further comprises a medium for containing the stem cells. In another embodiment, the medium comprises at least one growth factor selected from a group consisting of transforming growth factor-β, insulin-like growth factor, platelet derived growth factor, epidermal growth factor, acidic fibroblast growth factor, basic fibroblast growth factor, and hepatocytic growth factor. In still another embodiment, the medium comprises at least one nutrient selected from a group consisting of vitamin A, retinoic acid, vitamin B series, and vitamin C.

Some aspects of the present invention provide a delivery instrument for delivering an umbrella-configured breast matrix to a breast of a patient comprising: a hollow tubular sheath having a distal tip, a lumen having an opening at the distal tip, and a handle portion; a plunger inside the lumen, wherein the plunger is activated by a pushing mechanism located at the handle portion, and wherein the lumen is sized and configured for appropriately receiving an umbrella-configured breast matrix at a collapsed profile.

Some aspects of the invention provide a delivery instrument for delivering an umbrella-configured breast matrix to a breast of a patient comprising a tubular applicator having a distal tip, a distal portion and a handle portion, wherein the distal portion is sized and configured for appropriately receiving an umbrella-configured breast matrix at a collapsed profile over the distal portion. In one embodiment, the umbrella-configured breast matrix is characterized by a plurality of radially expandable extending elements, each extending element having a distal end and a proximal end, wherein the proximal ends from all extending elements are secured together at one point, and wherein the umbrella-configured breast matrix further comprises at least one connecting member between any two extending elements, wherein the connecting member is selected from a group consisting of netting, strings, threads, porous membranes, and porous biodegradable films.

Some aspects of the invention provide a breast template for delivering stem cell formulation or composition to a breast defect of a patient, comprising a flexible band or apparatus with at least one throughput hole for guiding an injecting needle to penetrate into a breast of the patient, wherein the flexible band or apparatus is configured to be placed intimately against a surface of the breast.

### BRIEF DESCRIPTION OF THE DRAWINGS

Additional objects and features of the present invention will become more apparent and the disclosure itself will be best understood from the following Detailed Description of the Exemplary Embodiments, when read with reference to the accompanying drawings.

FIG. 1 shows a schematic diagram of a method for treating a breast defect.

FIG. 2 shows an anatomic illustration of a woman breast.

FIG. 3 shows a breast implant embodiment of the fishbone design: (A) an expanded profile, and (B) a collapsed profile.

FIG. 4 shows a first breast implant embodiment of the umbrella design: (A) a delivery instrument, (B) an expanded device profile, and (C) a collapsed device profile.

FIG. 5 shows a second breast implant embodiment of the umbrella design: (A) a delivery instrument, (B) a proximal cross-sectional view, (C) a distal cross-sectional view, and (D) an expanded device profile.

FIG. 6 shows a breast implant of the wrap-around design: (A) an expanded profile, (B) a collapsed profile, and (C) a simulated profile.

FIG. 7 shows a breast implant of the yo-yo design.

FIG. 8 shows a perspective view of a breast template that guides the injection loci of the needle penetration into breast tissue.

FIG. 9 shows a cross-sectional view of the breast template that is flexible to fit a range of breast sizes.

FIG. 10 shows a perspective view of placing the breast template onto a breast of the patient.

FIG. 11 shows one embodiment of components of an injecting needle.

FIG. 12 shows an illustrative view of the injecting needle.

FIG. 13 shows a cross-sectional view of the injecting needle of FIG. 12.

FIG. 14 shows steps A-C for releasing the stem cell formulation in situ.

FIG. 15 shows a system for delivering stem cell formulation to a patient.

### DETAILED DESCRIPTION OF THE EXEMPLARY EMBODIMENTS

The preferred embodiments of the present invention described below relate particularly to methods and a composition for the differentiation and culture of adipose tissue-derived stromal cells into breast tissue cells. The cells produced by the methods of the invention are useful in providing a source of fully differentiated and functional cells for tissue regeneration for the treatment of human breast defect, repair and augmentation. Thus, in one aspect, the invention provides a method for differentiating adipose tissue-derived stromal cells into breast tissue cells comprising culturing stromal cells in a composition that comprises a medium capable of supporting the growth and differentiation of stromal cells into functional breast cells. This invention further provides methods for the introduction and position of these stromal cells in breast defect areas for repair or augmentation. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

By “progenitor” it is meant an oligopotent or multipotent stem cell which is able to divide without limit and, under specific conditions, can produce daughter cells which terminally differentiate such as into breast cells. These cells can be used for transplantation into a heterologous, autologous, or non-autologous host. By heterologous is meant a host other than the animal from which the progenitor cells were originally derived. By autologous is meant the identical host from which the cells were originally derived. Cell suspensions in culture medium are supplemented with certain specific growth factor that allows for the proliferation of target progenitor cells and seeded in any receptacle capable of sustaining cells, though as set out above, preferably in culture flasks or roller bottles. Cells typically proliferate within 3-4 days in a 37° C. incubator, and proliferation can be reinitiated at any time after that by dissociation or purification of the cells and re-suspension in fresh medium containing specific growth factors. The medium for cells suspension is also considered one type of cell carriers.

By “adipose” is meant any fat tissue. The adipose tissue may be brown or white adipose tissue, derived from subcutaneous, omental/visceral, mammary, gonadal, or other adipose tissue site. A convenient source of adipose tissue is from liposuction surgery, however, the source of adipose tissue or the method of isolation of adipose tissue is not critical to the invention. When stromal cells are desired for autologous transplantation into a subject, the adipose tissue will be isolated from that subject and administered to the specific breast defect site for tissue regeneration.

Liposuction is the most frequently performed procedure in plastic surgery. Liposuction or suction-assisted lipectomy removes fat cells from parts of the body where excess fat cells exist. The liposuction procedure involves making one or more small poke wounds in areas like the abdomen, hips or thighs. Through these small incisions, a long metal tube (a cannula), with small holes at one end and connected to one atmosphere of negative pressure at the other end, is inserted. The cannula, in the 3-5 mm diameter range, is repeatedly moved in and out of the surgical site. A network of holes, like a sponge or Swiss cheese, is made in the bulging area and the fat is liquefied and removed. Sometimes, ultrasound vibrational energy is added to enhance the fat emulsification (ultrasound-assisted liposuction). Afterwards, the overlying skin is compressed with a binder or girdle to tighten the tissues for a couple of weeks. During the procedure, the area to be suctioned is filled with saline solution, local anesthetic, and vasoconstrictor. The saline serves to emulsify and soften the fat and makes it easier to remove.

Any medium capable of supporting stromal cells in tissue culture may be used, for example, Dulbecco's Modified Eagle's Medium that supports the growth of fibroblasts. Growth factors are generally added to the medium for supporting stromal cells in tissue culture. Typically, 0 to 20% Fetal Bovine Serum (FBS) is added to the above medium in order to support the growth of stromal cells. The cells could be incubated at a temperature around 37° C. with the carbon dioxide content maintained between 1% to 10% and the oxygen content between 1% and 20%.

Non-limiting examples of media useful in the methods of the invention can contain fetal serum of bovine or other species at a concentration of at least 1% to about 30%, preferably at least about 5% to 15%, mostly preferably about 10%. Embryonic extract of chicken or other species can be present at a concentration of about 1% to 30%, preferably at least about 5% to 15%, most preferably about 10%.

The growth factors of the invention may include, but not limited to, transforming growth factor-β (TGF-β1, TGF-β2, TGF-β3 and the like), insulin-like growth factor, platelet derived growth factor, epidermal growth factor, acidic fibroblast growth factor, basic fibroblast growth factor, hepatocytic growth factor, and the like. The concentration of growth factors is about 1 to about 100 ng/ml. In one embodiment, the matrix for incorporating the stromal cells is a component of the collagenous extracellular matrix such as collagen I (particularly in the form of a gel). Other nutrient, such as vitamin A, vitamin A analogue (such as retinoic acid), vitamin B series, vitamin C, and vitamin C analogue or other vitamins may be added to the medium. The concentration of retinoic acid or other nutrient is about 0.1 to about 10 μg/ml.

The present invention also provides a method for formulating adipose derived stromal cells, either after in vitro culture or in absence of in vitro culture, with a biocompatible pharmaceutical carrier for injecting into the breast of a subject. In one embodiment, the biocompatible carrier may be in the form of slurry, gel, a malleable gel, colloid, solution, or suspension. A process for manufacturing an implantable cells-seeded gel material may comprise the steps of: providing a biocompatible carrier and stem cells source; combining the cells and the carrier in a uniformly suspended form; and applying a pressurizing force to the combined fluid for either injecting into the breast of the subject or for collapsing into a malleable gel before administering into the breast.

The adipose tissue derived stromal cells useful in the methods of invention may be isolated by a variety of methods known to those skilled in the art. For example, such methods are described in U.S. Pat. No. 6,153,432 incorporated herein in its entirety. In a preferred method, adipose tissue is isolated from a mammalian subject, preferably a human subject. A preferred source of adipose tissue is omental adipose. In humans, the adipose is typically isolated by liposuction. If the cells of the invention are to be transplanted into a human subject, it is preferable that the adipose tissue be isolated from that same subject so as to provide for an autologous transplant. Alternatively, the administered tissue may be allogenic.

Stem cells also provide promise for improving the results of gene therapy. A patient's own stem cells could be genetically altered in vitro, then reintroduced in vivo to produce a desired gene product. These genetically altered stem cells would have the potential to be induced to differentiate to form a multitude of cell types for implantation at specific sites in the body, or for systemic application. Alternately, heterologous stem cells could be genetically altered to express the recipient's major histocompatibility complex (MHC) antigen, or no MHC, to allow transplant of those cells from donor to recipient without the associated risk of rejection. The cells produce therapeutic enzymes, proteins, or other products in the human so that genetic defects are corrected. A method of using the cells for gene therapy in a subject in need of therapeutic treatment, involving genetically altering the cells by introducing into the cell an isolated pre-selected DNA encoding a desired gene product, expanding the cells in culture, and introducing the cells into the body of the subject to produce the desired gene product. Some aspects of the invention provide genetically altered stem cells to form a multitude of cell types for implantation at a breast in the body for treating a patient with prior breast cancer or tumor.

The present invention provides a method of repairing damaged tissue in a human subject in need of such repair by expanding the isolated multipotent adult stem cells in culture, and contacting an effective amount of the expanded cells with the damaged tissue of the subject. The cells may be introduced into the body of the subject by localized injection. The cells may be introduced into the body of the subject in conjunction with a suitable matrix scaffold. The matrix scaffold may provide additional genetic material, cytokines, growth factors, or other factors to promote growth and differentiation of the cells. The cells may be encapsulated or co-mixed prior to introduction into the body of the subject, such as with a polymer capsule or other biodegradable substrate.

In one embodiment of the invention, an adipose tissue derived stromal cell induced to express at least one phenotypic characteristic of a neuronal, astroglial, hepatic, hematopoietic, or breast tissue cell is provided. Phenotypic markers of the desired cells are well known to those of ordinary skill in the art, and copiously published in the literature. Additional phenotypic markers continue to be disclosed or can be identified without undue experimentation. Any of these markers can be used to confirm that the adipose cell has been induced to a differentiated state. Lineage specific phenotypic characteristics can include cell surface proteins, cytoskeletal proteins, cell morphology, and secretory products. Some aspects of the invention provide adipose tissue-derived stromal cells that exhibit the improved properties of increased extracellular matrix proteins and/or a lower amount of lipid than a mature isolated adipocyte.

Malson et al. in U.S. Pat. No. 4,772,419, entire contents of which are incorporated herein by reference, describes a crosslinked hyaluronic acid (or salt thereof) gel material that may be formed into a shaped article by pressure-drying or freeze-drying. The crosslinked hyaluronic material may be stored dry, and implanted or placed upon a body in dry form, or alternatively after being rehydrated in a saline solution. The crosslinking present in the material causes the material to be rehydrated as a sponge or foam, wherein the structure or shape is maintained, rather than forming a flowable hydrogel or putty. Some aspects of the invention provide a crosslinked gel material as a shaped article loaded with adipose-derived stem cells or progenitor breast tissue cells.

In another embodiment, the biocompatible cell carrier (for example, for cells to home in) or matrix may be a shaped construct, structure, or 3-dimensional scaffold. Examples of biocompatible carrier material includes alginate, agarose, fibrin, collagen, chitosan, gelatin, elastin, and combinations thereof. In one embodiment, the biocompatible cell carrier is biodegradable or bioresorbable. Examples of biodegradable matrix material may include, but not limited to, polymers or copolymers of lactide, glycolide, caprolactone, polydioxanone, and trimethylene carbonate. Examples of biodegradable matrix material may also include polyorthoesters and polyethylene oxide.

Further examples of biodegradable polymers for construction of the matrix may include aliphatic polyesters, alginate, cellulose, chitin, chitosan, collagen, copolymers of glycolide, copolymers of lactide, elastin, fibrin, glycolide/l-lactide copolymers (PGA/PLLA), glycolide/trimethylene carbonate copolymers (PGA/TMC), glycosaminoglycans, hydrogel, lactide/tetramethylglycolide copolymers, lactide/trimethylene carbonate copolymers, lactide/ε-capro-lactone copolymers, lactide/σ-valerolactone copolymers, l-lactide/dl-lactide copolymers, methyl methacrylate-N-vinyl pyrrolidone copolymers, modified proteins, nylon-2 PHBA/γ-hydroxyvalerate copolymers (PHBA/HVA), PLA/polyethylene oxide copolymers, PLA-polyethylene oxide (PELA), poly(amino acids), poly(trimethylene carbonates), poly hydroxyalkanoate polymers (PHA), poly(alklyene oxalates), poly(butylene diglycolate), poly(hydroxy butyrate) (PHB), poly(n-vinyl pyrrolidone), poly(ortho esters), polyalkyl-2-cyanoacrylates, polyanhydrides, polycyanoacrylates, polydepsipeptides, polydihydropyrans, poly-dl-lactide (PDLLA), polyesteramides, polyesters of oxalic acid, polyglycolide (PGA), polyiminocarbonates, polylactides (PLA), poly-1-lactide (PLLA), polyorthoesters, poly-p-dioxanone (PDO), polypeptides, polyphosphazenes, polysaccharides, polyurethanes (PU), polyvinyl alcohol (PVA), poly-β-hydroxypropionate (PHPA), poly-p-hydroxybutyrate (PBA), poly-σ-valerolact-one poly-β-alkanoic acids, poly-β-malic acid (PMLA), poly-ε-caprolactone (PCL), pseudo-Poly(Amino Acids), starch trimethylene carbonate (TMC), tyrosine based polymers. In another embodiment, the cell carrier or matrix functions as a reservoir for cell differentiation and controlled release to adjacent tissue sites.

Current protocols for differentiating isolated human preadipocytes into adipocytes can be performed by a variety of methods, for example, the preadipocyte cell component in human adipose tissue (the so-called “stromal vascular fraction” or SVF) can be isolated using collagenase treatment. The isolated human preadipocytes can then be driven to differentiate into adipocytes by a variety of chemical treatments. For example, Hauner's laboratory (Journal Clin Invest., (1989) 34:1663-1670) has shown that human preadipocytes can be induced to differentiate in serum-free medium containing 0.2 nM triiodothyronine, 0.5 μM insulin and 0.1 μM glucocorticoid. Similarly, it is disclosed in U.S. Pat. No. 4,153,432, entire contents of which are incorporated herein by reference, for the differentiation of human preadipocytes that incubating isolated human preadipocytes, plated at least about 25,000 cells/cm², in a medium containing, glucose, a cyclic AMP inducer such as isobutylmethylxanthine or forskolin, a glucocorticoid or glucocorticoid analogue, insulin or an insulin analogue and a PPARγ agonist or a RXR agonist.

One aspect of the invention discloses co-administration of stem cells and gene transfer for therapeutic vasculogenesis, wherein viability of stem cells could be further enhanced with endothelial precursor cells (EPCs), which are capable of generating blood vessels. The same methodology could be used to modulate neo-vascularization in breast augmentation process of the invention. For instance, treatment with the EPCs that carried an angiogenic protein enhances blood vessel formation. In a series of related studies, circulating EPCs have been shown to be mobilized endogenously in response to tissue ischemia or exogenously by cytokine therapy, after which they augmented the neo-vascularization of ischemic tissues (Nat Med. 1999;5:434-438). Implantation of bone marrow mononuclear cells into ischemic myocardium in swine enhanced collateral perfusion and regional myocardial function (J Am Coll Cardiol. 2001;37:1726-1732). This therapeutic angiogenesis may have been due to the natural ability of the bone marrow cells to secrete potent angiogenic ligands and cytokines, as well as to be incorporated into foci of neo-vascularization (Circulation. 2001;104:1046-1052). Other observations have shown that EPCs prevented cardiomyocyte apoptosis, reducing remodeling and improving cardiac function in areas of neo-vascularized ischemic myocardium in rats (Nat Med. 2001;7:430-436).

An important issue concerning the therapeutic use of stem cells is the quantity of cells and cells colony necessary to achieve an optimal effect. In current human studies of autologous mononuclear bone marrow cells, empirical doses of 10 to 40×10⁶ are being used with encouraging results. In a study designed to treat peripheral vascular disease with autologous bone marrow, much larger doses were administered to the gastrocnemius muscle (2.7×10⁹ cells), with minimal inflammation and positive results (Lancet. 2002;360:427-435).

Importantly, endothelial cells may derive from circulating stem cells (Science 1997;275:964-967). For instance, the implantation of expanded CD34⁺ endothelial progenitor cells has the capacity to induce angiogenesis. It was reported that incubation of mouse neural stem cells with human endothelial yielded the lining of blood vessels. Specifically, notable fraction of the stem cells was showing the biochemical and structural characteristics of endothelial cells. A team led by Dr. Silviu Itescu of Columbia U. has reported that certain bone marrow cells (cells that express the c-Kit protein) can be manipulated to become angioplasts. When injected into the tails of rats with simulated heart attacks, these highly differentiated cells helped regenerate blood vessel tissue (Nature Medicine in April 2001). Viable method of controlled neo-angiogenesis includes application of sustain-released forms of angiogenic growth factors, for example, encapsulating angiogenic growth factors within a biodegradable matrix or capsule.

### EXAMPLE NO. 1

### Methods of Transplantation

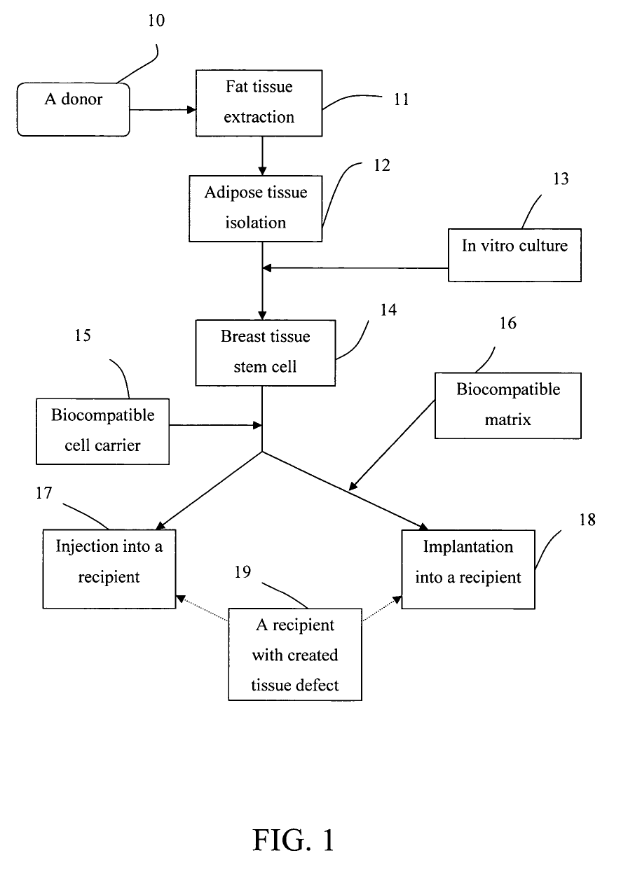

FIG. 1 shows a method of treating a breast defect in a patient, the method comprising: a) differentiating an isolated human adipose tissue derived stromal cell into a breast tissue cell; and b) administering the breast tissue cell to a breast defect area in the patient. In one embodiment, the fat tissue from the donor is further differentiated into adipocytes in an in vitro procedure, followed by isolation to obtain a concentrated substance of breat tissue cells prior to the step of administering. In one embodiment, the breast tissue defect is created as an adjunct step for promoting stem cells differentiation and tissue regeneration at about the defect site.

As shown in FIG. 1, the fat tissue extraction step 11 may be carried out, for example by liposuction from a donor 10. The adipose tissue isolation step 12 may include breakup of the fat mass and removal of the unwanted non-cellular material. In vitro culture step 13 may be optional; however, nutrients, growth factors and other substance may be added to enhance cell differentiation into breast tissue cells. In one embodiment, the breast tissue cells 14 can be formulated with biocompatible cell carrier 15 for injection into a recipient 17. In another embodiment, the breast tissue cells 14 can be further deposited onto a biocompatible matrix 16 for implantation into a recipient 18. It is one object of the present invention to provide a recipient 19 with created tissue defect enabling the stem cells tissue regeneration via the injection route 17 or the implantation route 18.

In another embodiment of the invention, support cells are used to promote the differentiation of the adipose-derived stromal cells prior to or following implantation into the defect breast site of a recipient. The support cells can be human or non-human animal derived cells. Adipose-derived cells are isolated and cultured within a population of cells; most preferably, the population is a defined population. The population of cells is heterogeneous and includes support cells for supplying factors to the progenitor or stem cells of the invention. Support cells include other cell types that will promote the differentiation, growth and maintenance of the desired cells. By way of illustration, adipose-derived stromal cells are first isolated by any of the means described above, and grown in culture in the presence of other support cells. In another embodiment, the support cells are derived from primary cultures of these cell types taken from cultured human organ tissue. In yet another embodiment, the support cells are derived from immortalized cell lines. In some embodiments, the support cells are obtained autologously.

### EXAMPLE NO. 2

### Cell Carriers and Matrix

The formula consisting of breast tissue cells and cell carriers can be injected to the defect site of the breast using a syringe or other fluid delivery apparatus. In one embodiment, the formula is intended to enhance revascularization in situ. In another embodiment, the formula is intended to promote growth or multiplication of fat cells in the breast. For illustration purposes, the biocompatible matrix for cells to home in or adhere for intended differentiation purposes may comprise a foam or sponge that is compressible for inserting into the breast with a small opening. The biocompatible foam or sponge construct is characterized with plural pores, wherein at least a portion of the pores is interconnected and open to the outside of the construct. The foam or sponge can be cut, sized, and shaped as an implant. In one embodiment, the formula consisting of breast tissue cells and cell carriers may be loaded on the biocompatible matrix/foam before matrix/foam delivery into a recipient. Alternatively, the formula consisting of breast tissue cells and cell carriers may be injected by a needle to about the matrix/form site after the matrix/foam is implanted in place.

Several needle types are feasible for injecting the formula or formulation into the target breast site for tissue regeneration or augmentation. The needle may have a curved or straight tip and a sharp stylet that would protrude past the needle tip to facilitate perforation of the skin. The needle is usually about 15 gauges (14 to 17 gauges) or with metal wings attached to the needle hub. Among them, the Huber point needle has a long, sharp, curved tip designed to lessen the pain of an injection and decrease the risk of depositing plugs of skin into underlying tissues.

The advantages of the Huber non-coming needle or substantially equivalent others to be utilized in the invention show that the end of the needle is curved or hooded, and “shrouds” the coming end to eliminate severing tissue in a 360 degree circle. The end of the needle may be equipped with a simple plug that could be removed and replaced from inside the outer needle or rotated to an open (180 degree rotation) position, like the gates on the side of the needle apparatus.

Breast Template

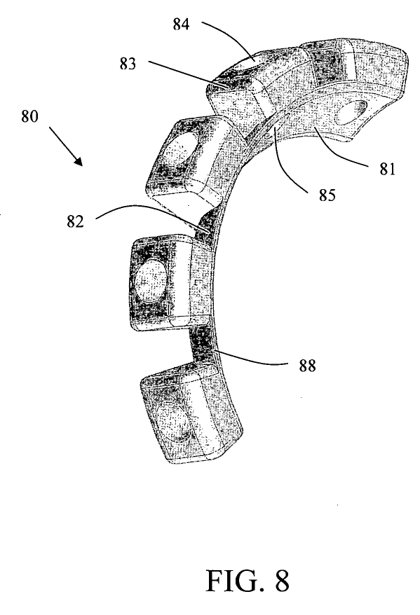

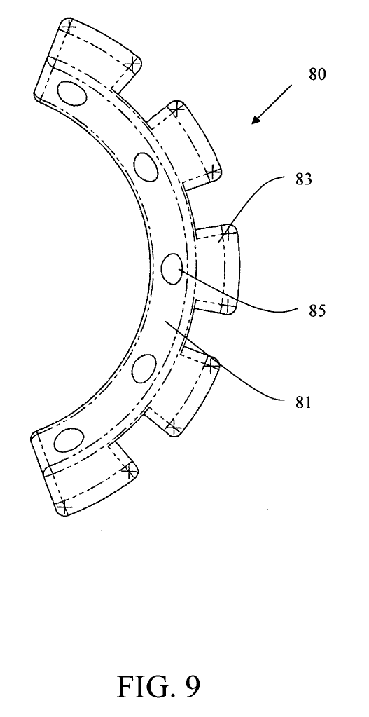

Some aspects of the invention relate to a breast template that guides an injecting needle to position the needle outlet port(s) at a desired location for delivering stem cells formulation. The stem cells formulation may include the stem cells derived from adipose tissue, growth factors, and biodegradable cell carriers. FIG. 8 shows a perspective view of a breast template 80 that guides the injection loci of the needle penetration into breast tissue. FIG. 9 shows a cross-sectional view of the breast template 80 that is flexible and may have different sizes to fit a range of breast sizes or contours. In one embodiment, the template 80 comprises a flexible elongate band or apparatus 88 with certain thickness having an inner surface 81 to be placed against the breast skin and an exterior surface 82 facing a physician. The elongate band is sized and configured for intimately resting on the breast skin of a patient. The exterior side of the elongate band comprises a plurality of protrusions 83, each protrusion having a throughput hole for inserting the needle. The throughput hole of the protrusion 83 has a needle entrance port 84 and needle exit port 85 that are sized appropriately for guided penetration. In one embodiment, the throughput hole is configured at an angle with respect to the inner surface 81 and the exterior surface 82 for guided penetration.

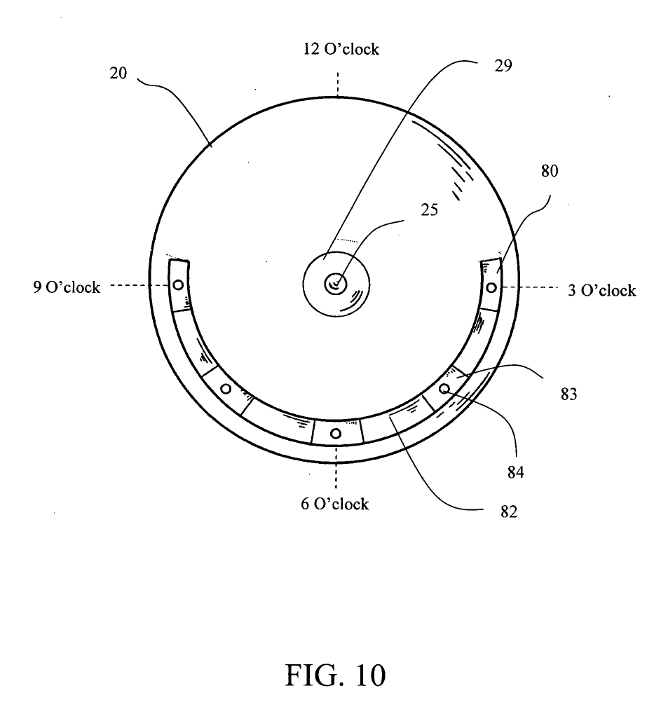

FIG. 10 shows a perspective view of placing the breast template 80 onto a breast 20 of the patient. As shown, the breast template 80 may be placed at lower half or lower quarter of the breast. The needle entrance port 84 of the protrusion 83 is used to guide the needle to penetrate into the breast at the front side or the rear side of the nipple 25. Upon gradually removing the needle, the stem cell formulation (or composition) can be released from the needle outlet port(s). In one embodiment, the breast template is a ring-like complete circle so as to finish the delivery of stem cell formulation by placing the ring-like breast template only once against the target breast. In another embodiment, the breast temperate covers one-half or a quarter of the breast periphery.

Tissues are highly organized in their geometry and architecture with respect to how cells are positioned relative to each other, as well as to the surrounding soluble factors and extracellular matrix molecules within a given microenvironment. The engineered microscale biomaterials may be formulated into 3D cellular microenvironment to generate homogeneous microtissues by controlling the spatial distribution of cells and molecules within hydrogels and directly engineer the microvasculature into 3D structures.

The syringe can be a double barrel mixing element that mixes the slurry (or cell carrier with growth factors) with the progenitor cells (or subcutaneous fat stem cells with enough angiogenesis factors) in a spiral barrel so the mixing is substantially homogeneous and complete when the mixed substrate exits the distal end of the syringe.

The gel or foam of the present invention may comprise methylcellulose, a temperature-sensitive polymer. Methylcellulose (MC) is a water-soluble polymer derived from cellulose, the most abundant polymer in nature. As a viscosity-enhancing polymer, it thickens solutions without precipitation over a wide pH range. A novel method using a temperature-sensitive polymer (Methylcellulose) to thermally gel aqueous alginate blended with distinct salts (CaCl₂, Na₂HPO₄, or NaCl), as a pH-sensitive hydrogel was developed for protein drug delivery (Biomacromolecules 2004;5:1917-1925). In the preparation of cells loaded hydrogels herein, it is suggested that stem cells is well-mixed to the dissolved aqueous methylcellulose or methylcellulose/alginate blended with salts at 4° C. and then gel by elevating the temperature to 37° C. In one embodiment, the blend (stem cells or adipose-derived breast tissue progenitor cells plus aqueous methylcellulose) is injected into the breast of a recipient and become a gel in situ because of the body temperature at 37° C., a characteristic temperature for methylcellulose.

All methylcellulose compositions exhibit the classical physical behavior of cellulose ethers, changing from a solution at lower temperature to a gel at elevated temperatures. When exposing methylcellulose to an increasing temperature, the methylcellulose shows an initial period of relatively constant viscosity. Then the solution undergoes an abrupt increase in viscosity at a characteristic temperature corresponding to initiation of the first gelation phenomenon. The temperature at which gelation is initiated can be altered by varying a number of factors, including concentration of methylcellulose polymer, formulation of the aqueous solvent, additives, and heating rate. Methylcellulose was reported biocompatible with little toxicity due to degraded byproducts (Biomaterials 2001;22:1113-1123). It was reported that injectable methylcellulose appears to be a suitable scaffold for bridging traumatically injured tissue when a cavity forms within the first few days following a traumatic insult to the cortex.

Poly(N-isopropyl acrylamide) demonstrated a fully expanded chain conformation below 32° C. and a collapsed compact conformation at high temperatures (J Biomed Mater Res 1993;27:1243-1251). In one aspect of the invention, adipose-derived breast tissue progenitor cells or stem cells are mixed with poly(N-isopropyl acrylamide) to form an injectable gel material. After loading the gel material into the breast of a recipient at adjacent the porous scaffold, the gel material collapses and squeezes into the pores of the scaffold, where the stem cells start differentiation and proliferation to repair or treat breast tissue defect.

The currently available breast augmentation devices include silicone breast implants filled with silicone fluid/gel or saline. Their disadvantages include: prone to rupture, prone to leak, losing the shape, or interfering with mammograms. Herein it is one object of the present invention to provide a breast implant that maintains its shape, will repair itself continuously, biocompatible, requires a minimally invasive intervention for implantation, and presents minimal or no complications because of its biocompatibility. In one embodiment, the breast implant of the present invention comprises a main shaping framework with sustained resorbable or biodegradable stent lattice, supplemented by a biodegradable or bioresorbable polymer netting. The material may include hydrogel with scaffold, matrix or foam configuration.

The delivery system of the presentation is catheter based. In one embodiment, the ability to repair itself continuously comprises delivering cell seeding slurry or concentrated cultured stem cells that is programmed to develop fat cells to augment the breast defect, wherein the cells may be derived from bone marrow stem cells or omentum fat cells.

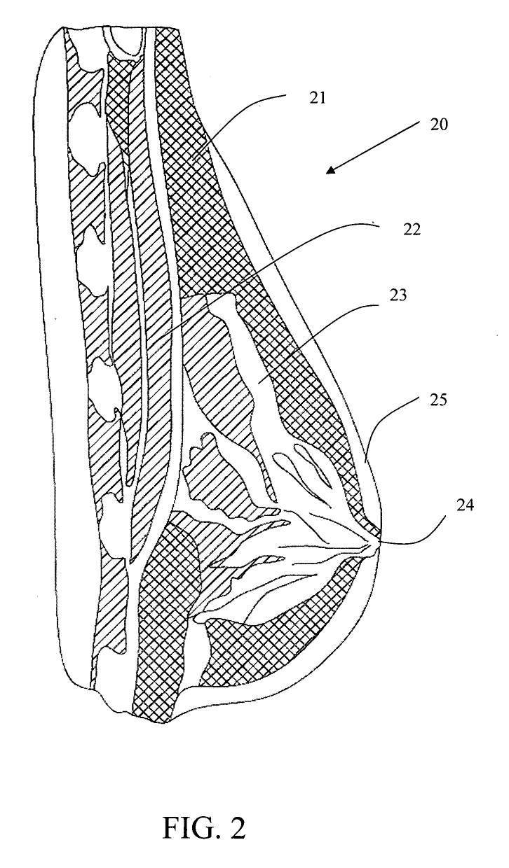

Some aspects of the present invention relate to various breast implant embodiments with stem cells loaded configuration or post-implantation stem cells receivable configuration and delivery systems thereof. FIG. 2 shows an anatomic illustration of a woman breast 20 comprising fatty tissue 21, muscle 22, ducts 23, and a nipple 24, among others. One embodiment of the present invention is to deliver a breast implant through the nipple and extendably follow the duct 23 or the space under the subcutaneous layer 25 of the breast. Another embodiment of the delivery route is similar to that of the silicone-gel breast implant placement.

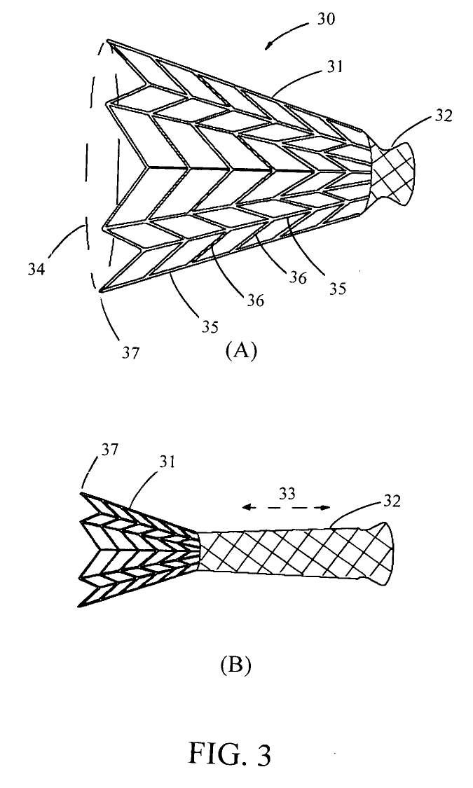

Various design configurations of the breast implant or scaffolds for stem cells seeding and eventual differentiation/regeneration/proliferation in situ are illustrated in FIGS. 3-7. FIG. 3 shows a breast implant embodiment of the fishbone design 30 at (A) an expanded profile, and (B) a collapsed profile. In general, the fishbone implant 30 comprises an expandable construct 31 with a plurality of close cells 38 formed between the longitudinal elements 35 and the connecting transverse elements 36, wherein the construct 31 is enclosed and loaded within the lumen of a sheath 32 during the initial delivery phase, the sheath being unobstructively movable substantially along the same direction 33 of the construct 31. In one embodiment, the sheath 32 is a solid cylinder, a meshed cylinder, or a flexible tubular apparatus that is detachable from the construct at the end of the delivery phase. The distal ends 37 of the construct 31 form a circular shape 34 after the implant is delivered to the breast site of a recipient. As discussed before, stem cells or adipose-derived breast tissue progenitor cells may be loaded on the breast implant before delivery or injected to adjacent the implant after the implant is delivered in place.

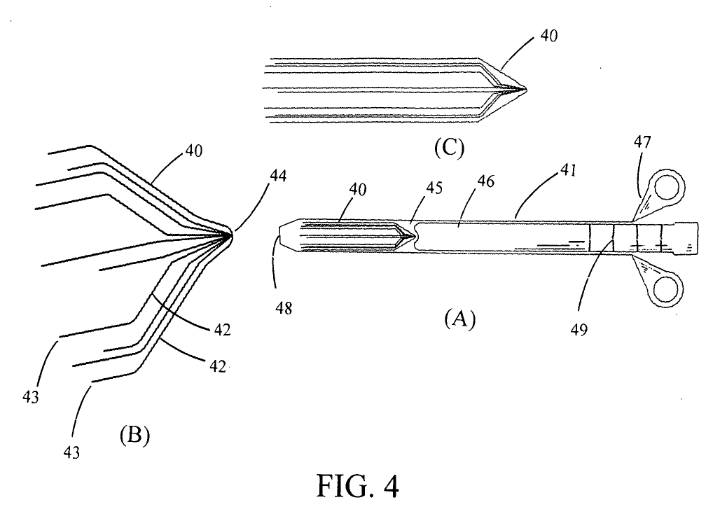

FIG. 4 shows a first breast implant embodiment of the umbrella design 40 with (A) a delivery instrument 41, (B) at an expanded device profile, and (C) at a collapsed device profile. In general, the umbrella implant 40 comprises a plurality of radially expandable extending elements 42, each having a distal end 43 and a proximal end 44. In one embodiment, the proximal ends 44 of all extending elements are secured together at one point. The delivery instrument 41 may comprise a lumen 45 with a pushing plunger 46, wherein the plunger is activated by a pushing mechanism 47 located at the handle of the delivery instrument. In operations, the needle tip 48 of the delivery instrument 41 contacts or partially penetrates the nipple of a recipient, the plunger is activated until the umbrella implant is fully deployed, which is indicated by a pre-marked marker 49 on the delivery instrument 41.

Some aspects of the invention provide a delivery instrument for delivering an umbrella-configured breast matrix to a breast of a patient comprising: a hollow tubular sheath having a distal needle tip, a lumen having an opening at the distal tip, and a handle portion; a plunger inside the lumen, wherein the plunger is activated by a pushing mechanism located at the handle portion; and wherein the lumen is sized and configured for appropriately receiving an umbrella-configured breast matrix at a collapsed profile.

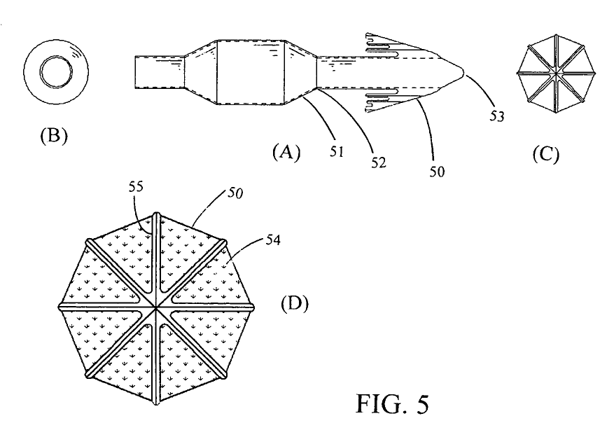

Alternatively, FIG. 5 shows a second breast implant embodiment of the umbrella design 50 with (A) a delivery instrument 51, (B) a proximal cross-sectional view of the delivery instrument, (C) a distal cross-sectional view of the delivery instrument, and (D) at an expanded device profile. Instead of loading the breast implant in the lumen of a delivery instrument as shown in FIG. 4, the breast implant 50 is loaded outside of the delivery instrument 51 in FIG. 5. In one embodiment, the second umbrella implant 50 comprises extending elements 55 and some connecting members 54 between the elements 55 to form an umbrella shape. The connecting member 54 may comprise netting, strings, threads, porous membranes, porous biodegradable films, biocompatible polymers, etc. as disclosed above. In operations, the instrument tip 53 of the delivery instrument 51 contacts and penetrates into the nipple of a recipient, the instrument 51 is pushed forward until the umbrella implant 50 at its collapsed profile is fully deployed inside the breast, which is indicated by a pre-marked marker 52 on the delivery instrument 51.

Some aspects of the invention provide a delivery instrument for delivering an umbrella-configured breast matrix to a breast of a patient comprising a tubular applicator having a distal tip, a distal portion and a handle portion, wherein the distal portion is sized and configured for appropriately receiving an umbrella-configured breast matrix at a collapsed profile over the distal portion.

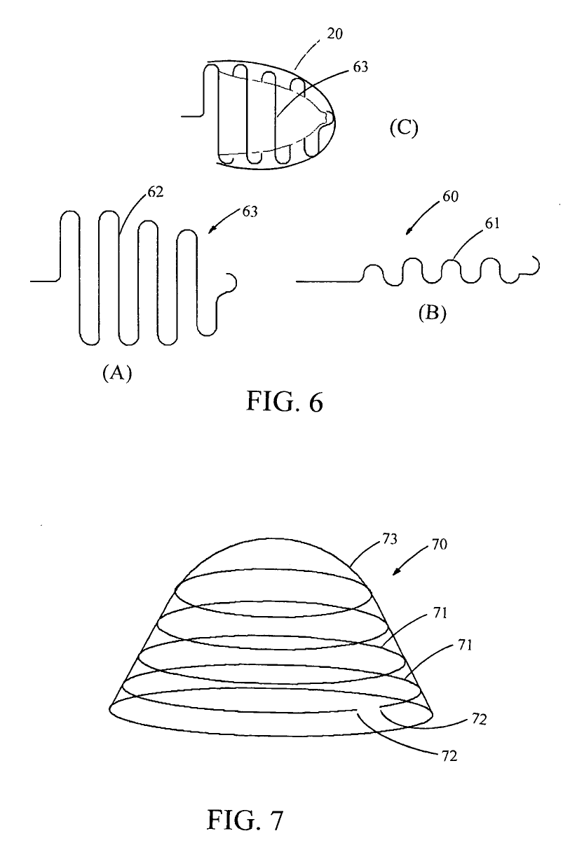

FIG. 6 shows a breast implant of the wrap-around design 60, (A) at an expanded profile, (B) at a collapsed profile, and (C) with a simulated profile. The wrap-around implant 60 may be made of shape memory polymer, shape memory biodegradable polymer or shape memory alloy, such as Nitinol. In one embodiment, a pre-shaped implant 60 is sized and shaped as a straight wire with a few small curvatures 61 at a first configuration (as shown in FIG. 6B). The implant is delivered into the breast, say from the nipple. After the implant is in place, the implant 60 is changed to a second 3-D configuration (as shown in FIG. 6A) with a few large curvatures 62 by the shape memory characteristics, mostly by raising the implant temperature to pass a shape transition temperature of the building material. In one embodiment, the shape transition temperature is configured to be a few degrees, preferably 1 to 5° C., above the body temperature. In operations, at least a major portion of the implant in the second configuration 63 is placed at a space under the subcutaneous layer of the breast (as shown in FIG. 6C) serving as a scaffold for stem cell deposition/differentiation leading to cell proliferation and tissue regeneration.

FIG. 7 shows a breast implant of the yo-yo design 70. In one embodiment, the implant comprises a plurality of circular rings 71 with varying diameters. In another embodiment, at least a portion of the circular rings 71 is an open ring with two ends 72 so that the ring can be inserted into the breast by first entering one end of the open ring into the nipple. In an alternate embodiment, at least two rings are releasably secured to each other by a circular semi-ring 73 to form a bowl-like configuration. In operations, each component (the circular rings or the circular semi-ring) of the yo-yo implant occupies certain locations of the breast for intended tissue regeneration by the loaded adipose-derived stem cells. In an alternative embodiment, the aforementioned breast implant is delivered to the breast by surgical operations or other penetration methods so that the implant serves as the supporting matrix for stem cells to repair or augment a breast tissue defect in a patient. The “breast implant” herein is intended to mean a scaffold, matrix, or stent to partially support the breast and partially support the loaded stem cells composition for tissue repair/augmentation, whereas the breast implant does not herein include or indicate any breast silicone prosthesis.

Cells Injecting Needle Configuration

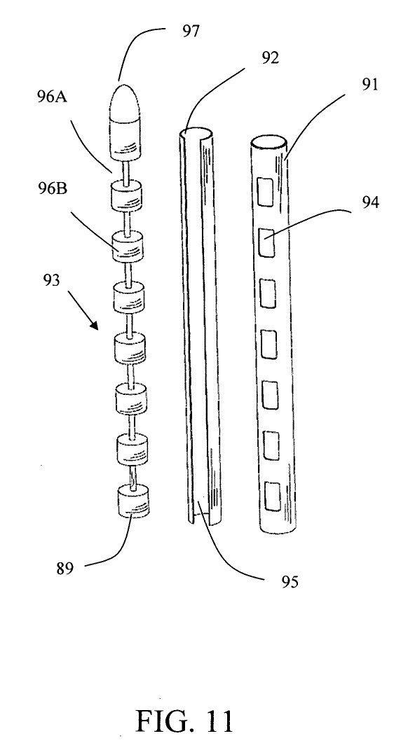

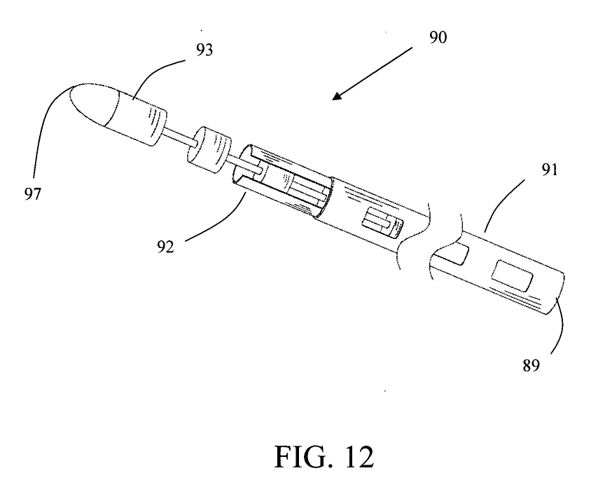

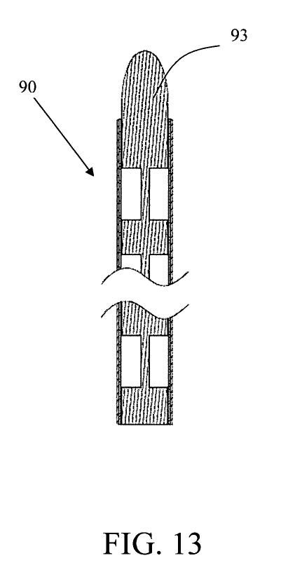

FIG. 11 shows one embodiment of several components of an injecting needle 90, whereas FIG. 12 and FIG. 13 show an illustration view and a cross-sectional view of the injecting needle, respectively. In one embodiment, the needle of the invention 90 comprises an outer tubular member 91 having a fluid passageway connected to at least one opening 94 that is sized and configured for releasing the stem cell formulation from the fluid passageway at a desired location inside the breast and a desired timing. The needle further comprises an intermediate tubular member 92 having a slit opening 95 that is sized and configured for releasing the stem cell formulation. Further, the needle comprises an inner elongate member 93 having a distal end 97, at least one recess section 96A and at least one circular expanded section 96B that fits inside the inner void of the intermediate member 92. The distal end 97 may be sharpened to penetrate into the breast skin. The clearance between the circular section 96B and the inner void is sized and configured for rotating the inner member 93 effortlessly relative to the intermediate member 92, but preventing any leakage from one recess section to an adjacent recess section. In one embodiment, the inner elongate member 93 further comprises a central lumen or passageway that has at least one opening at each recess section and is in fluid communication through a proximal port 89 to an outside pressure source (not shown) or a pressurized supply of stem cell formulation.

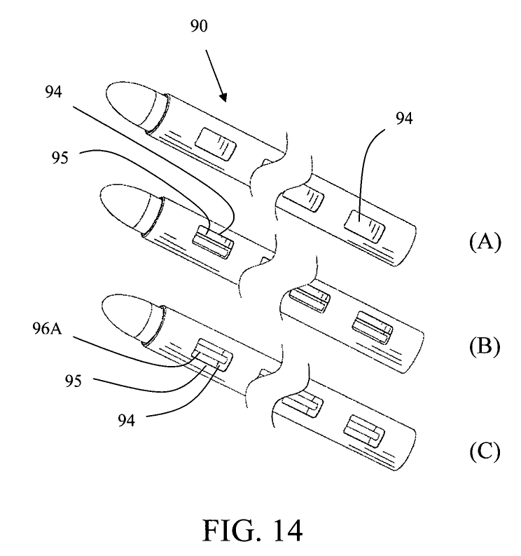

FIG. 14 shows steps A to C for releasing the stem cell formulation in situ from the needle to a breast tissue site. As described above, the three members of the injecting needle are sized and configured to be concentrically rotatable relative to each other. In FIG. 14A, the opening 94 of the outer tubular member 91 does not match with the slit opening 95 of the intermediate member 92. Therefore, no fluid communication passes through the opening 94. In FIG. 14B, the opening 94 of the outer tubular member 91 matches with the slit opening 95 of the intermediate member 92, but does not match with the recess section 96A of the inner member. Again, there is no fluid communication passing through the opening 94. In FIG. 14C, at least a portion of the slit opening 95 and the recess section 96A matches or in-lines with the opening 94 of the outer member. With some external pressure through the central lumen of the inner member 93, the stem cell formulation could be released in situ from the needle to a breast tissue site.

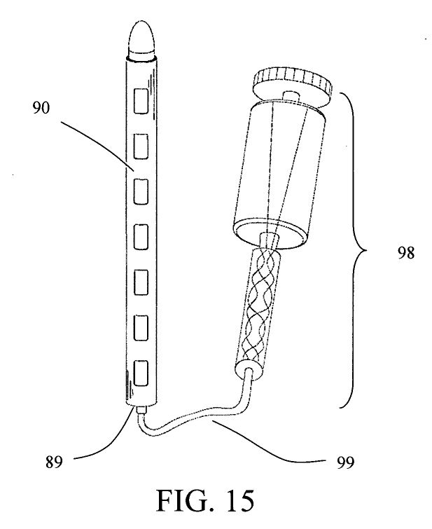

FIG. 15 shows a system for delivering stem cell formulation or composition to a patient. In one embodiment, the system comprises the needle portion 90 and a syringe-like supplier 98 of the stem cell formulation that is connected through a flexible connecting means 99 to the proximal port 89 of the needle portion 90. In another embodiment, the system comprises the needle portion 90 and an external pressure source that is configured to push the stem cell formulation out of the recess section 96A of the inner member 93 to the surrounding tissue.

Although the present invention has been described with reference to specific details of certain embodiments thereof, it is not intended that such details should be regarded as limitations upon the scope of the invention. Many modifications and variations are possible in light of the above disclosure.

What is claimed is:

1. A breast template for delivering stem cell formulation to a breast defect of a patient, comprising a flexible band with at least one throughput hole for guiding an injecting needle to penetrate into a breast of the patient, wherein the flexible band is configured to be placed intimately against a surface of the breast.

2. The breast template of claim 1, wherein stem cells of the stem cell formulation are derived from adipose tissue.

3. The breast template of claim 2, wherein the stem cells comprise breast tissue progenitor cells.

4. The breast template of claim 2, wherein the stem cells comprise subcutaneous fat stem cells with enough angiogenesis factors.

5. The breast template of claim 2, wherein the stem cells are genetically altered stem cells adapted for gene therapy.

6. The breast template of claim 1, wherein the stem cell formulation comprises lipocytes or adipocytes that are stromal only with no ductal or lobular developmental potential.

7. The breast template of claim 1, wherein the stem cell formulation further comprises a breast matrix.

8. The breast template of claim 7, wherein the breast matrix is biodegradable or bioresorbable.

9. The breast template of claim 7, wherein the breast defect is traumatically created by inserting said breast matrix into the breast of the patient.

10. The breast template of claim 8, wherein the breast matrix encapsulates at least one angiogenic growth factor.

11. The breast template of claim 1, wherein the stem cell formulation comprises a medium.

12. The breast template of claim 11, wherein the medium comprises at least one angiogenic growth factor.

13. The breast template of claim 11, wherein the medium comprises at least one growth factor selected from a group consisting of transforming growth factor-β, insulin-like growth factor, platelet derived growth factor, epidermal growth factor, acidic fibroblast growth factor, basic fibroblast growth factor, and hepatocytic growth factor.

14. The breast template of claim 11, wherein the medium comprises at least one nutrient selected from a group consisting of vitamin A, retinoic acid, vitamin B series, and vitamin C.

15. The breast template of claim 1, wherein the injecting needle comprises a delivery port that is pressurized by an external pressure source for releasing said stem cell formulation to said breast defect.

16. The breast template of claim 1, wherein the injecting needle is between about 14 and 17 gauges.

17. The breast template of claim 1, wherein the injecting needle has a long, sharp, curved tip configured to lessen the pain of an injection and decrease the risk of depositing plugs of skin into underlying tissues.

18. The breast template of claim 1, wherein the stem cell formulation comprises endothelial precursor cells adapted for modulating neo-vascularization in a breast augmentation process.

19. The breast template of claim 1, wherein the breast template covers one-half or a quarter of a breast periphery.

20. The breast template of claim 1, wherein the breast temperate is a ring-like template.
